(19) United States
(12) Patent Application Publication
(10) Pub. No.: US 2012/0115222 A1
(43) Pub. Date: May 10, 2012

(54) COMPOSITION AND METHOD FOR MAINTENANCE, DIFFERENTIATION, AND PROLIFERATION OF STEM CELLS

(75) Inventors: Sherry L. Voytik-Harbin, Zionsville, IN (US); Edward F. Srour, Indianapolis, IN (US); Melissa A. Kacena, Zionsville, IN (US)

(73) Assignee: PURDUE RESEARCH FOUNDATION, West Lafayette, IN (US)

(21) Appl. No.: 13/383,796

(22) PCT Filed: Jul. 16, 2010
(86) PCT No.: PCT/US10/42290

§ 371 Date: Jan. 12, 2012

Related U.S. Application Data
(60) Provisional application No. 61/226,092, filed on Jul. 16, 2009.
(60) Provisional application No. 61/230,041, filed on Jul. 30, 2009.

(51) Int. Cl. C12N 5/077; C12N 5/02; C12N 5/07

(52) U.S. Cl. 435/347; 435/325; 435/373; 435/375

(57) ABSTRACT

Compositions and methods for the proliferation, differentiation, and maintenance of stem cells are described. Preferred are the use of hematopoietic stem cells in combination with a collagen matrix.

### CROSS REFERENCE TO RELATED APPLICATIONS

This application claims priority under 35 U.S.C. §119(e) to U.S. Provisional Application Nos. 61/226,092, filed Jul. 16, 2009, and 61/230,041, filed Jul. 30, 2009, each of which is expressly incorporated by reference herein.

### FIELD OF THE INVENTION

This invention relates to compositions and methods for the maintenance, proliferation, and differentiation of hematopoietic stem cells.

### BACKGROUND AND SUMMARY

Adult stem cells can be found throughout the body in highly specialized microenvironments, called niches. Within each niche, the proliferation and differentiation of the resident stem cells is controlled via three main pathways: cell-cell interactions, cell-matrix interactions, and soluble factors [1,2].

In vertebrates, the hematopoietic stem cell (HSC) niche is known to be located within the marrow of long bones. Bone marrow transplants take advantage of this fact: the marrow is relatively rich in HSCs, and can thus be used to reconstitute a patient's immune system after aggressive cancer treatments, specifically radiation treatment or chemotherapy.

As a whole, bone marrow is a poorly characterized material, being composed of dozens of cell types and many soluble factors [4]. In addition, the intraosteal region contains a huge range of matrix components, from soft, fatty marrow to blood vessels to rigid fibers of cancellous bone. As such, it is not immediately clear which factors within this environment are necessary for HSC maintenance.

Compositions and methods for the proliferation and phenotypic maintenance of HSCs are described herein. Engineered collagen matrices with varied microstructure-mechanical properties and methods for maintaining HSC proliferation (self-renewal) and function (clonogenic potential and in vivo marrow repopulation capacity) are described. The ability to expand viable HSC in an ex vivo environment has tremendous clinical applications, specifically with regards to recovery from cancer treatments. In addition, this system will provide insight into the development of blood cancers and other pathologies.

The following embodiments of the invention are contemplated:

1. A method for maintaining or proliferating hematopoietic stem cells in vitro, said method comprising the steps of:

contacting an hematopoietic stem cell population with an engineered collagen matrix; and

maintaining or proliferating the hematopoietic stem cells in vitro.

2. The method of clause 1 wherein the collagen matrix has a fibril density of about 5% to about 30%, and wherein the collagen matrix has a storage modulus of about 10 Pa to about 3200 Pa.

3. The method of clause 1 or 2 further comprising the step of contacting said matrix with osteoblasts.

4. The method of clause 3 wherein the osteoblasts enhance proliferation or maintenance of the hematopoietic stem cells.

5. The method of any one of clauses 1 to 4 wherein the matrix has a storage modulus of about 10 Pa to about 100 Pa.

6. The method of any one of clauses 1 to 4 wherein the matrix has a storage modulus of about 500 Pa to about 2000 Pa.

7. The method of any one of clauses 1 to 4 or 6 wherein the matrix has a storage modulus of about 700 Pa to about 1500 Pa.

8. The method of any one of clauses 1 to 4 or 6 to 7 wherein the matrix has a storage modulus of about 700 Pa to about 900. Pa.

9. The method of any one of clauses 1 to 4 or 6 to 8 wherein the hematopoietic stem cells are undifferentiated.

10. The method of any one of clauses 1 to 4 or 6 to 9 wherein the hematopoietic stem cell population exhibits expression of an hematopoietic stem cell marker.

11. The method of any one of clauses 1 to 4 or 6 to 10 wherein the hematopoietic stem cell population exhibits expression of an hematopoietic stem cell function.

12. The method of any one of clauses 1 to 4 or 6 to 11 wherein the hematopoietic stem cell population exhibits clonogenic potential.

13. The method of any one of clauses 1 to 4 or 6 to 12 wherein the hematopoietic stem cell population exhibits in vivo marrow repopulating potential.

14. The method of any one of clauses 1 to 4 or 6 to 13 for use in maintaining the hematopoietic stem cells in an undifferentiated state for implantation into a patient in need of treatment with the hematopoietic stem cells.

15. The method of any one of clauses 1 to 14 for use in drug efficacy or toxicity testing wherein the hematopoietic stem cells are contacted with a drug.

16. The method of any one of clauses 1 to 5 for use in producing a population of proliferating hematopoietic stem cells or their progeny for implantation of the cells into a patient.

17. The method of clause 14 further comprising the step of contacting the matrix with hyaluronic acid.

18. The method of clause 16 wherein the matrix comprises a mixture of type I and type III collagen.

19. A composition comprising hematopoietic stem cells and an engineered collagen matrix.

20. The composition of clause 19 further comprising osteoblasts.

21. The composition of clause 19 or 20 wherein the collagen matrix has a fibril density of about 5% to about 30%, and wherein the collagen matrix has a storage modulus of about 10 Pa to about 3200 Pa.

22. The composition of any one of clauses 20 to 21 wherein the osteoblasts enhance proliferation or function of the hematopoietic stem cells.

23. The composition of any one of clauses 19 to 22 wherein the matrix has a storage modulus of about 10 Pa to about 100 Pa.

24. The composition of any one of clauses 19 to 22 wherein the matrix has a storage modulus of about 500 Pa to about 2000 Pa.

25. The composition of any one of clauses 19 to 22 or 24 wherein the matrix has a storage modulus of about 700 Pa to about 900 Pa.

26. The composition of any one of clauses 19 to 22 or 24 to 25 wherein the hematopoietic stem cells are undifferentiated.

27. The composition of any one of clauses 19 to 22 or 24 to 26 wherein the hematopoietic stem cell population exhibits expression of an hematopoietic stem cell marker.

28. The composition of any one of clauses 19 to 22 or 24 to 27 wherein the hematopoietic stem cell population exhibits expression of an hematopoietic stem cell function.

29. The composition of any one of clauses 19 to 22 or 24 to 28 wherein the hematopoietic stem cell population exhibits clonogenic potential.

30. The composition of any one of clauses 19 to 22 or 24 to 29 wherein the hematopoietic stem cell population exhibits in vivo marrow repopulating potential.

31. The composition of any one of clauses 19 to 22 or 24 to 29 for use in maintaining the hematopoietic stem cells in an undifferentiated state for implantation of the cells into a patient.

32. The composition of any one of clauses 19 to 31 for use in drug toxicity or drug efficacy testing.

33. The composition of any one of clauses 19 to 23 for use in producing a population of proliferating hematopoietic stem cells for implantation into a patient.

34. The composition of any one of clauses 19 to 22 or 24 to 32 further comprising hyaluronic acid.

35. The composition of any one of clauses 19 to 23 or 32 to 33 wherein the matrix comprises a mixture of type I and type III collagen.

36. Use of hematopoietic stem cells and an engineered collagen matrix for maintaining or proliferating hematopoietic stem cells in vitro.

37. The use of clause 36 wherein the collagen matrix has a fibril density of about 5% to about 30%, and wherein the collagen matrix has a storage modulus of about 10 Pa to about 3200 Pa.

38. The use of clause 36 or 37 wherein osteoblasts are added to the matrix and the hematopoietic stem cells.

39. The use of clause 38 wherein the osteoblasts enhance proliferation of the hematopoietic stem cells.

40. The use of any one of clauses 36 to 39 wherein the matrix has a storage modulus of about 10 Pa to about 100 Pa.

41. The use of any one of clauses 36 to 39 wherein the matrix has a storage modulus of about 500 Pa to about 2000 Pa.

42. The use of any one of clauses 36 to 39 or 41 wherein the matrix has a storage modulus of about 700 Pa to about 900 Pa.

43. The use of any one of clauses 36 to 39 or 41 to 42 wherein the hematopoietic stem cells are undifferentiated.

44. The use of any one of clauses 36 to 39 or 41 to 43 wherein the hematopoietic stem cell population exhibits expression of an hematopoietic stem cell marker.

45. The use of any one of clauses 36 to 39 or 41 to 44 wherein the hematopoietic stem cell population exhibits expression of an hematopoietic stem cell function.

46. The use of any one of clauses 36 to 39 or 41 to 45 wherein the hematopoietic stem cell population exhibits clonogenic potential.

47. The use of any one of clauses 36 to 39 or 41 to 46 wherein the hematopoietic stem cell population exhibits in vivo marrow repopulating potential.

48. The use of any one of clauses 36 to 39 or 41 to 44 wherein the hematopoietic stem cells or their progeny are maintained in an undifferentiated state for implantation of the cells into a patient.

49. The use of any one of clauses 36 to 48 in drug toxicity or drug efficacy testing.

50. The use of any one of clauses 36 to 40 to produce a population of proliferating hematopoietic stem cells for implantation of the cells into a patient.

51. The use of clause 48 wherein hyaluronic acid is added to the matrix.

52. The use of clause 50 wherein the matrix comprises a mixture of type I and type III collagen.

53. The method of any one of clauses 1 to 13 further comprising the step of diagnosing a patient with a blood cell disease.

54. The composition of any one of clauses 19 to 31 wherein the composition is for use in diagnosing a patient with a blood cell disease.

55. The use of any one of clauses 36 to 48 in a diagnostic test for a blood cell disease.

56. A method for differentiating hematopoietic stem cells in vitro, said method comprising the steps of:

contacting an hematopoietic stem cell population with an engineered collagen matrix; and

differentiating the hematopoietic stem cells in vitro.

57. The method of clause 56 wherein the collagen matrix has a fibril density of about 5% to about 30%, and wherein the collagen matrix has a storage modulus of about 10 Pa to about 3200 Pa.

58. The method of clause 56 or 57 further comprising the step of contacting said matrix with osteoblasts.

59. The method of clause 58 wherein the osteoblasts enhance proliferation of the hematopoietic stem cells.

60. The method of any one of clauses 56 to 59 wherein the matrix has a storage modulus of about 10 Pa to about 100 Pa.

61. The method of any one of clauses 56 to 60 for use in drug efficacy or toxicity testing wherein the hematopoietic stem cells are contacted with a drug.

62. The method of any one of clauses 56 to 60 for use in producing a population of proliferating hematopoietic stem cells or their progeny for implantation of the cells into a patient.

63. The method of any one of clauses 56 to 62 wherein the matrix comprises a mixture of type I and type III collagen.

64. The method of any one of clauses 56 to 63 wherein the hematopoietic stem cells are differentiated down a specific lineage.

### BRIEF DESCRIPTION OF THE DRAWINGS

FIG. 1 shows a diagram of the hematopoietic stem cell (HSC) niche.

FIG. 2 shows Lin⁻Sca⁺c-Kit⁻ (LSK) cell Proliferation (Panel A) and colony forming unit (CFU) fold increase (Panel B).

FIG. 3 shows hematopoietic stem cell (HSC) clonogenic potential.

FIG. 4 shows Lin⁻Sca⁺c-Kit⁻ (LSK) cell phenotype maintenance.

FIG. 5 shows Lin⁻Sca⁺c-Kit⁻ (LSK) cell phenotype maintenance.

FIG. 6 shows LSK cell production in pig skin collagen matrices in which type I collagen (200 Pa) was co-polymerized with type III collagen (0.5 mg/ml) or HA (1 mg/ml). LSK and OB were seeded at 625 cells/well and 25,000 cells/well, respectively.

FIG. 7 shows flow cytometry results illustrating the effect of fibril density and matrix stiffness on LSK proliferation, CFU fold increase, plating efficiency, and Lin−Sca1+ marker maintenance for: a 200 Pa LSK culture (5B, top panels); a traditional 2D LSK culture (5A, top panels); a traditional 2D culture with LSK+OB (5A, middle panels); an 800 Pa 3D culture with LSK+OB (5A, bottom panels); a 3D collagen type I and type III matrix with LSK+OB culture (5B, middle panels); and a 3D collagen type I and hyaluranic acid matrix with an LSK+OB culture (5B, bottom panels).

FIG. 8 shows confocal reflection microscopy-obtained 3D image stacks of matrices prepared with pig-skin collagen (A) as well as the oligomer-rich (B) and monomer-rich (C) fractions (scale bar=50 μm).

FIG. 9 upper panels show changes in G′, δ, and Ec for matrices (0.7 mg/ml collagen concentration) prepared with varied AMW for both single and pooled sources. FIG. 9 lower panels show that matrices prepared with oligomer or monomer fractions demonstrate distinct fibril microstructure-mechanical behavior (all data points n=5).

FIG. 10 shows the colony forming potential of LSK in the presence or absence of OB within oligomer and reduced-oligomer matrices.

### DETAILED DESCRIPTION OF ILLUSTRATIVE EMBODIMENTS

As used herein “engineered collagen matrix” means a matrix that is polymerized in vitro under predetermined conditions selected from the group consisting of, but not limited to, pH, phosphate concentration, temperature, buffer composition, ionic strength, and composition and concentration of the collagen. An “engineered collagen matrix” can be made from purified collagen or partially purified extracellular matrix components.

As used herein “partially purified extracellular matrix components” are extracellular matrix components that are solubilized from intact extracellular matrix material wherein the collagen in the “partially purified extracellular matrix components” is not substantially free from impurities.

As used herein “purified collagen” is collagen that is substantially free of impurities (e.g., collagen that is 95% to 99.9% pure).

As used herein “engineered purified collagen matrix” means a purified collagen-based matrix that is polymerized in vitro under predetermined conditions selected from the group consisting of, but not limited to, pH, phosphate concentration, temperature, buffer composition, ionic strength, and composition and concentration of the collagen. An “engineered purified collagen matrix” is made from purified collagen.

As used herein “engineering a matrix” means polymerizing an “engineered collagen matrix” or an “engineered purified collagen matrix” in vitro.

As used herein “proliferating hematopoietic stem cells” means causing a population of hematopoietic stem cells or their progeny (e.g., myeloid and lymphoid lineages) to increase in number. In various aspects, the myeloid lineages can comprise monocytes, macrophages, neutrophils, basophils, eosinophils, erythrocytes, platelets, or dendritic cells. In other aspects, the lymphoid lineages can comprise T-cells, B-cells, or NK cells.

As used herein “maintaining hematopoietic stem cells” means maintaining the “sternness” of hematopoietic stem cells (i.e., maintaining the clonogenic potential of the stem cells as evidenced by the ability of the cells to produce colony-forming units as well as the capacity to repopulate the bone marrow in vivo). Hematopoietic stem cells that have maintained their “sternness” or their clonogenic potential are undifferentiated.

As used herein “differentiating hematopoietic stem cells” means causing the hematopoietic stem cells to progress to a myeloid or a lymphoid cell lineage. A myeloid lineage can comprise monocytes, macrophages, neutrophils, basophils, eosinophils, erythrocytes, platelets, or dendritic cells. A lymphoid lineage can comprise T-cells, B-cells, or NK cells.

As used herein “hematopoietic stem cells” means hematopoietic stem cells and associated progenitor cells. Hematopoietic stem cells can be identified and/or isolated based on specific cell markers (e.g., the Lineage−, Sca1⁺, and c-Kit+ hematopoietic stem cell markers) or specific functions characteristic of hematopoietic stem cells and known to those skilled in the art.

In one embodiment of the invention a method for maintaining or proliferating hematopoietic stem cells in vitro is provided. The method comprises the steps of contacting a hematopoietic stem cell population with an engineered collagen matrix, and maintaining or proliferating the hematopoietic stem cells in vitro. In another illustrative embodiment, a method for differentiating hematopoietic stem cells in vitro is provided. The method comprises the steps of contacting an hematopoietic stem cell population with an engineered collagen matrix, and differentiating the hematopoietic stem cells in vitro. In another embodiment, a composition comprising an engineered collagen matrix and hematopoietic stem cells is provided. In yet another embodiment, a use of hematopoietic stem cells and an engineered collagen matrix for maintaining or proliferating hematopoietic stem cells in vitro is provided. In any of these embodiments, osteoblasts can be present in combination with the hematopoietic stem cells. All of the embodiments described below apply to any embodiment described in this paragraph, or to any embodiment of the invention described in the Summary section of this application.

In any of the method, composition, or use embodiments described herein, the hematopoietic stem cells cultured on the engineered collagen matrix can maintain their clonogenic potential and can be used to repopulate bone marrow in vivo. In this embodiment, osteoblasts can be present in combination with the hematopoietic stem cells. In these embodiments, the hematopoietic stem cells are maintained in an undifferentiated state for injection or implantation into a patient in need of treatment with the hematopoietic stem cells (e.g., a patient in need of a bone marrow transplant).

In illustrative embodiments, hematopoietic stem cells can be cultured on engineered collagen matrices having a storage modulus of about 500 Pa to about 2000 Pa, about 700 Pa to about 1500 Pa, or about 700 Pa to about 900 Pa, resulting in maintenance of their clonogenic potential (for example, as evidenced by an increased ability to produce colony-forming units). In this illustrative embodiment, the hematopoietic stem cells can be cultured in vitro on the engineered collagen matrix, and then can be removed from the matrix, can be injected or implanted into a patient, and can be used to repopulate bone marrow in vivo, for example, in a patient in need of a bone marrow transplant.

In any of the method, composition, or use embodiments described herein, the differentiation (i.e., progression to a myeloid or a lymphoid cell lineage) or the proliferation (i.e., expansion) of the hematopoietic stem cells cultured on the engineered collagen matrix can be induced. In this embodiment, osteoblasts can be present in combination with the hematopoietic stem cells. In these embodiments, the hematopoietic stem cells are differentiated to form different cell lineages (e.g., myeloid and lymphoid lineages) by culturing the hematopoietic stem cells on an engineered collagen matrix with a specific composition. Myeloid lineages can comprise monocytes, macrophages, neutrophils, basophils, eosinophils, erythrocytes, platelets, or dendritic cells. Lymphoid lineages can comprise T-cells, B-cells, or NK cells.

In an illustrative embodiment, hematopoietic stem cells can be cultured on engineered collagen matrices with a storage modulus of about 10 Pa to about 100 Pa, resulting in differentiation (i.e., progression to a myeloid or a lymphoid cell lineage) and/or enhanced proliferation (i.e., expansion) of the hematopoietic stem cells compared to hematopoietic stem cells cultured on engineered collagen matrices with a storage modulus of, for example, about 500 Pa to about 2000 Pa, about 700 Pa to about 1500 Pa, or about 700 Pa to about 900 Pa. In this illustrative embodiment, the hematopoietic stem cells can be cultured in vitro on the engineered collagen matrix, and then can be removed from the matrix, and can be injected or implanted into a patient. For example, a patient in need of immunotherapy with specific mature blood cell types (e.g., antibody-producing lymphocytes) can be treated, or a patient in need of a transfusion with mature blood cells of a specific cell type can be treated.

In any of the method, composition, or use embodiments described herein, the engineered collagen matrices seeded with hematopoeitic stem cells can also be used in an in vitro model for drug efficacy or drug toxicity testing where the hematopoietic stem cells or their progeny (hematopoietic stem cells differentiated to a myeloid or a lymphoid cell lineage) are contacted with a drug. In this embodiment, osteoblasts can be present in combination with the hematopoietic stem cells. In this embodiment, the in vitro model for drug efficacy or toxicity testing is used to predict the clinical outcome of a drug used to treat blood diseases in the context of a myeloid or a lymphoid cell lineage or of undifferentiated hematopoietic stem cells cultured on engineered collagen matrices.

In any embodiment described herein, the engineered collagen matrix can be prepared by utilizing acid-solubilized collagen and defined polymerization conditions that are controlled to yield three-dimensional collagen matrices with a range of controlled assembly kinetics (e.g., polymerization half-time), molecular compositions, and fibril microstructure-mechanical properties, for example, as described in U.S. patent application Ser. Nos. 11/435,635 (published Nov. 22, 2007, as Publication No. 2007-0269476 A1) and 11/903,326 (published Oct. 30, 2008, as Publication No. 2008-0268052), each incorporated herein by reference.

In one aspect, purified collagen or partially purified extracellular matrix components can be used and can be obtained from a number of sources, including for example, porcine skin, to construct the engineered collagen matrices described herein. Suitable tissues useful as a collagen-containing source material for isolating collagen or extracellular matrix components to make the engineered collagen matrices described herein are submucosa tissues or any other extracellular matrix-containing tissues of a warm-blooded vertebrate. Suitable methods of preparing submucosa tissues are described in U.S. Pat. Nos. 4,902,508; 5,281,422; and 5,275,826, each incorporated herein by reference. Extracellular matrix material-containing tissues other than submucosa tissue may be used to obtain collagen in accordance with the methods and compositions described herein. Methods of preparing other extracellular matrix material-derived tissues for use in obtaining purified collagen or partially purified extracellular matrix components are known to those skilled in the art. For example, see U.S. Pat. Nos. 5,163,955 (pericardial tissue); 5,554,389 (urinary bladder submucosa tissue); 6,099,567 (stomach submucosa tissue); 6,576,265 (extracellular matrix tissues generally); 6,793,939 (liver basement membrane tissues); and U.S. patent application publication no. US-2005-0019419-A1 (liver basement membrane tissues); and international publication no. WO 2001/45765 (extracellular matrix tissues generally), each incorporated herein by reference. In various other embodiments, the collagen-containing source material can be selected from the group consisting of placental tissue, ovarian tissue, uterine tissue, animal tail tissue, and skin tissue. Any suitable extracellular matrix-containing tissue can be used as a collagen-containing source material to isolate purified collagen or partially purified extracellular matrix components.

An illustrative preparation method for preparing submucosa tissues as a source of purified collagen or partially purified extracellular matrix components is described in U.S. Pat. No. 4,902,508, the disclosure of which is incorporated herein by reference. In one embodiment, a segment of vertebrate intestine, for example, preferably harvested from porcine, ovine or bovine species, but not excluding other species, is subjected to abrasion using a longitudinal wiping motion to remove cells or cell-removal is accomplished by hypotonic or hypertonic lysis. In one embodiment, the submucosa tissue is rinsed under hypotonic conditions, such as with water or with saline under hypotonic conditions and is optionally sterilized. In another illustrative embodiment, such compositions can be prepared by mechanically removing the luminal portion of the tunica mucosa and the external muscle layers and/or lysing resident cells with hypotonic or hypertonic washes, such as with water or saline. In these embodiments, the submucosa tissue can be stored in a hydrated or dehydrated state prior to isolation of the purified collagen or partially purified extracellular matrix components. In various aspects, the submucosa tissue can comprise any delamination embodiment, including the tunica submucosa delaminated from both the tunica muscularis and at least the luminal portion of the tunica mucosa of a warm-blooded vertebrate.

In the various embodiments described herein, the purified collagen can also comprise exogenously added glycoproteins, proteoglycans, glycosaminoglycans (e.g., chondroitins and heparins), hyaluronic acid, etc. In this embodiment, hyaluronic acid can enhance maintenance of “sternness” of the hematopoietic stem cells (i.e., their clonogenic potential) in comparison to compositions where no exogenously added hyaluronic acid is present. In another embodiment, the partially purified extracellular matrix components can comprise glycoproteins, proteoglycans, glycosaminoglycans (e.g., chondroitins and heparins), hyaluronic acid, etc. extracted from the insoluble fraction with the collagen.

In various illustrative embodiments, the purified collagen or the partially purified extracellular matrix components or the engineered collagen matrices formed from these components can be disinfected and/or sterilized prior to seeding the matrices with hematopoietic stem cells, using conventional sterilization techniques including propylene oxide or ethylene oxide treatment, gas plasma sterilization, gamma radiation, electron beam, and/or peracetic acid sterilization. Sterilization techniques which do not adversely affect the structure and biotropic properties of the collagen can be used. Illustrative sterilization techniques are exposing the purified collagen or the partially purified extracellular matrix components or the engineered collagen matrices to peracetic acid, 1-4 Mrads gamma irradiation (or 1-2.5 Mrads of gamma irradiation), ethylene oxide treatment, or gas plasma sterilization. In one embodiment, the collagen-containing source material, the purified collagen, the partially purified extracellular matrix components, or the engineered collagen matrices can be subjected to one or more sterilization processes. In an illustrative embodiment, peracetic acid can be used for sterilization.

Typically, prior to extraction, the collagen-containing source material is comminuted by tearing, cutting, grinding, or shearing the collagen-containing source material. In one illustrative embodiment, the collagen-containing source material can be comminuted by shearing in a high-speed blender, or by grinding the collagen-containing source material in a frozen state (e.g., at a temperature of −20° C., −40° C., −60° C., or −80° C. or below prior to or during the comminuting step) and then lyophilizing the material to produce a powder having particles ranging in size from about 0.1 mm² to about 1.0 mm². In one illustrative embodiment, the collagen-containing source material is comminuted by freezing and pulverizing under liquid nitrogen in an industrial blender. In this embodiment, the collagen-containing source material can be frozen in liquid nitrogen prior to, during, or prior to and during the comminuting step.

In any of the illustrative embodiments described herein, after comminuting the collagen-containing source material, the material can be mixed (e.g., by blending or stirring) with an extraction solution to extract and remove soluble proteins. Illustrative extraction solutions include sodium acetate (e.g., 0.5 M and 1.0 M). Other methods for extracting soluble proteins are known to those skilled in the art and are described in detail in U.S. Pat. No. 6,375,989, incorporated herein by reference. Illustrative extraction excipients include, for example, chaotropic agents such as urea, guanidine, sodium chloride or other neutral salt solutions, magnesium chloride, and non-ionic or ionic surfactants.

In any illustrative aspect described herein, after the initial extraction, the soluble fraction can be separated from the insoluble fraction to obtain the insoluble fraction. For example, the insoluble fraction can be separated from the soluble fraction by centrifugation (e.g., 2000 rpm at 4° C. for 1 hour). In alternative embodiments, other separation techniques known to those skilled in the art, such as filtration, can be used. In one embodiment, the initial extraction step can be repeated one or more times, discarding the soluble fractions. In another embodiment, after completing the extractions, one or more steps can be performed of washing the insoluble fraction with water, followed by centrifugation, and discarding the supernatant.

In any of the embodiments described herein, the insoluble fraction can then be extracted (e.g., with 0.075 M sodium citrate) to obtain the purified collagen or the partially purified extracellular matrix components. In illustrative aspects the extraction step can be repeated multiple times retaining the soluble fractions. In one embodiment, the accumulated soluble fractions can be combined and can be clarified to form the soluble fraction, for example by centrifugation (e.g., 2000 rpm at 4° C. for 1 hour).

In any embodiment described herein, the soluble fraction can be fractionated to isolate the purified collagen, or the partially purified extracellular matrix components. In one illustrative aspect, the soluble fraction can be fractionated by dialysis. Suitable molecular weight cut-offs for the dialysis tubing or membrane are from about 3,500 to about 12,000 or about 3,500 to about 5,000 or about 12,000 to about 14,000. In various illustrative embodiments, the fractionation, for example by dialysis, can be performed at about 2° C. to about 37° C. for about 1 hour to about 96 hours. In one embodiment, the soluble fraction is dialyzed against a buffered solution (e.g., 0.02 M sodium phosphate dibasic). However, the fractionation can be performed at any temperature, for any length of time, and against any suitable buffered solution. In one embodiment, the precipitated collagen-containing material is then collected by centrifugation (e.g., 2000 rpm at 4° C. for 1 hour). In another embodiment, one or more steps can be performed of washing the collagen-containing material with water, followed by centrifugation, and discarding the supernatant.

In any of the embodiments described herein, the collagen-containing material can then be resuspended in an aqueous solution wherein the aqueous solution is acidic. For example, the aqueous acidic solution can be an acetic acid solution, but any other acids including hydrochloric acid, formic acid, lactic acid, citric acid, sulfuric acid, ethanoic acid, carbonic acid, nitric acid, or phosphoric acid can be used. For example, acids, at concentrations of from about 0.001 N to about 0.1 N, from about 0.005 N to about 0.1 N, from about 0.01 N to about 0.1 N, from about 0.05 N to about 0.1 N, from about 0.001 N to about 0.05 N, from about 0.001 N to about 0.01 N, or from about 0.01 N to about 0.05 N can be used to resuspend the collagen-containing material.

The term “lyophilized” means that water is removed from the composition, typically by freeze-drying under a vacuum. In one illustrative aspect, the isolated resuspended collagen-containing material can be lyophilized after it is resuspended for storage. In another illustrative embodiment, a matrix can be formed and the engineered collagen matrix itself can be lyophilized for storage. In one illustrative lyophilization embodiment, the resuspended collagen-containing material is first frozen, and then placed under a vacuum. In another lyophilization embodiment, the resuspended collagen-containing material can be freeze-dried under a vacuum. In another lyophilization embodiment, the collagen-containing material can be lyophilized before resuspension. Any method of lyophilization known to the skilled artisan can be used.

In any of the embodiments described herein, the acids described above can be used as adjuvants for storage after lyophilization in any combination. The acids that can be used as adjuvants for storage include hydrochloric acid, acetic acid, formic acid, lactic acid, citric acid, sulfuric acid, ethanoic acid, carbonic acid, nitric acid, or phosphoric acid, and these acids can be used at any of the above-described concentrations. In one illustrative embodiment, the lyophilizate can be stored (e.g., lyophilized in and stored in) an acid, such as acetic acid, at a concentration of from about 0.001 N to about 0.5 N or from about 0.01 N to about 0.5 N. In another embodiment, the lyophilizate can be stored in water with a pH of about 6 or below. In another embodiment, the lyophilized product can be stored dry. In other illustrative embodiments, lyoprotectants, cryoprotectants, lyophilization accelerators, or crystallizing excipients (e.g., ethanol, isopropanol, mannitol, trehalose, maltose, sucrose, tert-butanol, and tween 20), or combinations thereof, and the like can be present during lyophilization.

In any of the illustrative embodiments described herein, the collagen-containing material can be directly sterilized after resuspension, for example, with peracetic acid or with peracetic acid and ethanol (e.g., by the addition of 0.18% peracetic acid and 4.8% ethanol to the resuspended collagen-containing material before lyophilization). In another embodiment, sterilization can be carried out during the fractionation step. For example, the collagen-containing material can be dialyzed against chloroform, peracetic acid, or a solution of peracetic acid and ethanol (e.g., 0.18% peracetic acid and 4.8% ethanol) to disinfect or sterilize the material. The chloroform, peracetic acid, or peracetic acid/ethanol can be removed prior to lyophilization, for example by dialysis against an acid, such as 0.01 N acetic acid. In an alternative embodiment, the lyophilized composition can be sterilized directly after rehydration, for example, by the addition of 0.18% peracetic acid and 4.8% ethanol. In this embodiment, the sterilizing agent can be removed prior to polymerization of the collagen to form fibrils.

In any embodiment described herein, the collagen-containing material can be dialyzed against 0.01 N acetic acid, for example, prior to lyophilization to remove the sterilization solution and so that the collagen is in a 0.01 N acetic acid solution. In another embodiment, the collagen-containing material can be dialyzed against hydrochloric acid, for example, prior to lyophilization and can be lyophilized in hydrochloric acid and redissolved in hydrochloric acid, acetic acid, or water.

For use in producing the engineered collagen matrix that can be used for the maintenance, proliferation, or differentiation of hematopoietic stem cells or their progeny (e.g., myeloid lineages such as monocytes, macrophages, neutrophils, basophils, eosinophils, erythrocytes, platelets, or dendritic cells, and lymphoid lineages such as T-cells, B-cells, or NK cells), the redissolved lyophilizate can be subjected to varying conditions (e.g., pH, phosphate concentration, temperature, buffer composition, ionic strength, and composition and concentration of the purified collagen (dry weight/ml) or partially purified extracellular matrix components (dry weight/rill)) that result in polymerization to form an engineered collagen matrix with specific characteristics.

In any of the illustrative embodiments described herein, as discussed above, the polymerization reaction for the engineered collagen matrices can be conducted in a buffered solution using any biologically compatible buffer system known to those skilled in the art. For example, the buffer may be selected from the group consisting of phosphate buffer saline (PBS), Tris(hydroxymethyl)aminomethane Hydrochloride (Tris-HCl), 3-(N-Morpholino) Propanesulfonic Acid (MOPS), piperazine-n,n′-bis(2-ethanesulfonic acid) (PIPES), [n-(2-Acetamido)]-2-Aminoethanesulfonic Acid (ACES), N-[2-hydroxyethyl]piperazine-N′-[2-ethanesulfonic acid] (HEPES) and 1,3-bis[tris (Hydroxymethyl)methylamino]propane (Bis Tris Propane). In one embodiment the buffer is PBS, Tris, or MOPS and in one embodiment the buffer system is PBS, and more particularly 10×PBS. In accordance with one embodiment, the 10×PBS buffer at pH 7.4 comprises the following ingredients:

### 1.37 M NaCl

### 0.027 M KCl

### 0.081 M Na₂HPO₄

### 0.015 M KH₂PO₄

### 5 mM MgCl₂

55.5 mM glucose
All of the conditions that can be varied to polymerize and engineer the matrices described herein (e.g., pH, phosphate concentration, temperature, buffer composition, ionic strength, and composition and concentration of the collagen-containing material (dry weight/ml)) are described in U.S. application Ser. No. 11/903,326 (published Oct. 30, 2008, as Publication No. 2008-0268052), incorporated herein by reference.

The purified collagen and the partially purified extracellular matrix components are derived from a collagen-containing source material and, in some embodiments, may contain glycoproteins, such as laminin and fibronectin, proteoglycans, such as serglycin, versican, decorin, and perlecan, and glycosaminoglycans. In one embodiment, the collagen in the collagen-containing source material can be purified or partially purified to isolate the collagen using protocols known to the skilled artisan. In these embodiments, the purified collagen can be about 95%, about 96%, about 97%, about 98%, or about 99% pure, for example. In other embodiments, the purified collagen can be from about 95% to about 99.9% pure, from about 96% to about 99.9% pure, or from about 97% to about 99.9% pure. In yet another illustrative embodiment, the phrase “purified collagen” means the isolation of collagen in a form that is substantially free from impurities (e.g., typically the total amount of other components present in the composition represents less than 5%, or more typically less than 0.1%, of total dry weight). In an alternate embodiment, purified collagen can be purchased from sources such as Sigma Chemical Co. (St. Louis, Mo.), Advanced BioMatrix, Inc. (San Diego, Calif.), or Nutacon (Leimuiden, Netherlands).

As discussed, the engineered collagen matrices as herein described may be made under controlled conditions to obtain particular mechanical properties. For example, the engineered collagen matrices may have desired collagen fibril density, pore size (fibril-fibril branching), elastic modulus, tensile strain, tensile stress, linear modulus, compressive modulus, loss modulus, fibril area fraction, fibril volume fraction, collagen concentration, cell seeding density, shear storage modulus (G′ or elastic (solid-like) behavior), and phase angle delta (6 or the measure of the fluid (viscous)- to solid (elastic)-like behavior; δ equals 0° for Hookean solid and 90° for Newtonian fluid).

As used herein, a “modulus” can be an elastic or linear modulus (defined by the slope of the linear region of the stress-strain curve obtained using conventional mechanical testing protocols; i.e., stiffness), a compressive modulus, a loss modulus, or a shear storage modulus (e.g., a storage modulus). These terms are well-known to those skilled in the art.

As used herein, a “fibril volume fraction” (i.e., fibril density) is defined as the percent area of the total area occupied by fibrils in three dimensions.

As used herein, tensile or compressive stress “a” is the force carried per unit of area and is expressed by the equation:

$$σ - \begin{matrix}P \\ A\end{matrix} - \begin{matrix}P \\ ab\end{matrix}$$

where:
   s—stress
   P=force
   A—cross-sectional area
   a—width
   h—height

The force (P) produces stresses normal (i.e., perpendicular) to the cross section of the part (e.g., if the stress tends to lengthen the part, it is called tensile stress, and if the stress tends to shorten the part, it is called compressive stress).

As used herein, “tensile strain” is the strain caused by bending and/or stretching a material.

In any embodiment described herein, the fibril volume fraction of the matrix is about 1% to about 60%. In various embodiments, the engineered collagen matrix can contain fibrils with specific characteristics, for example, a fibril volume fraction (i.e., density) of about 2% to about 60%, about 2% to about 40%, about 5% to about 60%, about 15% to about 60%, about 2% to about 30%, about 5% to about 30%, about 15% to about 30%, or about 20% to about 30%.

In any of the illustrative embodiments described herein, the engineered collagen matrix can contain fibrils with specific characteristics, including, but not limited to, a modulus (e.g., a compressive modulus, loss modulus, or a storage modulus) of about 10 Pa to about 3200 Pa, about 10 Pa to about 700 Pa, about 10 Pa to about 300 Pa, about 10 Pa to about 200 Pa, about 10 Pa to about 100 Pa, about 500 Pa to about 2000 Pa, about 700 Pa to about 1500 Pa, about 700 Pa to about 900 Pa, or about 800 Pa.

In any of the embodiments described herein, the engineered collagen matrix can contain fibrils with specific characteristics, including, but not limited to, a phase angle delta (6) of about 0° to about 12°, about 0° to about 5°, about 1° to about 5°, about 4° to about 12°, about 5° to about 7°, about 8° to about 10°, and about 5° to about 10°.

In any of the illustrative embodiments described herein, qualitative and quantitative microstructural characteristics of the engineered collagen matrices can be determined by environmental or cryostage scanning electron microscopy, transmission electron microscopy, confocal microscopy, second harmonic generation multi-photon microscopy. In another embodiment, tensile, compressive and viscoelastic properties can be determined by rheometry or tensile testing. All of these methods are known in the art or are further described in U.S. patent application Ser. No. 11/435,635 (published Nov. 22, 2007, as Publication No. 2007-0269476 A 1), or are described in Roeder et al., J. Biomech. Eng., vol. 124, pp. 214-222 (2002), in Pizzo et al., J. Appl. Physiol., vol. 98, pp. 1-13 (2004), Fulzele et al., Eur. J. Pharm. Sci., vol. 20, pp. 53-61 (2003), Griffey et al., J. Biomed. Mater. Res., vol. 58, pp. 10-15 (2001), Hunt et al., Am. J. Surg., vol. 114, pp. 302-307 (1967), and Schilling et al., Surgery, vol. 46, pp. 702-710 (1959), incorporated herein by reference.

In another embodiment, a method for preparing the compositions described herein comprising an engineered collagen matrix and hematopoietic stem cells is provided. The compositions described herein can further comprise osteoblasts. In this embodiment, the method comprises the steps of engineering the matrix comprising collagen fibrils, and contacting the matrix with hematopoietic stem cells. The method can further comprise the step of contacting the matrix with osteoblasts.

Typically, the engineered collagen matrices are prepared from isolated collagen at collagen concentrations ranging from about 0.05 mg/ml to about 5.0 mg/ml, about 1.0 mg/ml to about 3.0 mg/ml, about 0.1 mg/ml to about 4.0 mg/ml, about 0.5 mg/ml to about 3.5 mg/ml, about 0.5 mg/ml to about 5.0 mg/ml, about 0.05 mg/ml to about 10 mg/ml, or about 0.05 to about 20 mg/ml, for example. In various illustrative embodiments, the collagen concentration is about 0.3 mg/ml, about 0.5 mg/ml, about 0.75 mg/ml, about 1.0 mg/ml, about 1.5 mg/ml, about 2.0 mg/ml, about 2.5 mg/ml, about 3.0 mg/ml, about 3.5 mg/ml, or about 5.0 mg/ml.

In any of these embodiments the engineered collagen matrix is seeded with hematopoietic stem cells (i.e., hematopoietic stem cells or hematopoietic progenitor cells). In another embodiment, the matrix can be further seeded with osteoblasts. In various embodiments, the engineered collagen matrix can be seeded with one or more cell types in combination. In illustrative embodiments, the osteoblasts can enhance proliferation, maintenance, or function of the hematopoietic stem cells.

As used herein, “stem cell” refers to an unspecialized cell from an embryo, fetus, or adult that is capable of self-replication or self-renewal and can develop into a variety of specialized cell types (i.e., potency). The term as used herein, unless further specified, encompasses oligopotent cells (those cells that can differentiate into a few cell types, e.g., lymphoid or myeloid lineages), and unipotent cells (those cells that can differentiate into only one cell type). Hematopoietic stem cells may be isolated from, for example, bone marrow, circulating blood, or umbilical cord blood by methods well-known to those skilled in the art. A cell marker can be used to select and purify the hematopoietic stem cells. For example, suitable markers are the Lin−, Sca1+, and c-Kit+ mouse or Lin−, CD34+, and c-Kit+ human hematopoietic stem cell markers. Cell markers may be used alone or in combination to select and purify the desired cell type for use in the compositions and methods herein described. The engineered collagen matrix can be seeded with autogenous cells isolated from the patient to be treated. In an alternative embodiment the cells may be xenogeneic or allogeneic in nature.

In any of the embodiments described herein, the hematopoietic stem cells are seeded on the engineered collagen matrix at a cell density of about 1×10⁶ to about 1×10⁸ cells/ml, or at a density of about 1×10³ to about 2×10⁶ cells/ml. In one embodiment stem cells are seeded at a density of less than 5×10⁴ cells/ml. In another embodiment cells are seeded at a density of less than 1×10⁴ cells/ml. In another embodiment, cells are seeded at a density selected from a range of about 1×10² to about 5×10⁶, about 0.3×10⁴ to about 60×10⁴ cells/ml, and about 0.5×10⁴ to about 50×10⁴ cells/ml. The cells are maintained, proliferated, or differentiated according to methods described herein or to methods well-known to the skilled artisan for cell culture.

In any of the various embodiments described herein, the engineered collagen matrices of the present invention can be combined, prior to, during, or after polymerization, with nutrients, including minerals, amino acids, sugars, peptides, proteins, vitamins (such as ascorbic acid), or glycoproteins that facilitate hematopoietic stem cell culture, such as laminin and fibronectin, hyaluronic acid, or growth factors such as platelet-derived growth factor, or transforming growth factor beta, and glucocorticoids such as dexamethasone. In other illustrative embodiments, fibrillogenesis inhibitors, such as glycerol, glucose, or polyhydroxylated compounds can be added prior to or during polymerization. In accordance with one embodiment, cells can be added to the purified collagen or the partially purified extracellular matrix components as the last step prior to the polymerization or after polymerization of the engineered collagen matrix. In other illustrative embodiments, cross-linking agents, such as carbodiimides, aldehydes, lysl-oxidase, N-hydroxysuccinimide esters, imidoesters, hydrazides, and maleimides, and the like can be added before, during, or after polymerization.

In any of the embodiments described herein, the cells may be isolated from the matrix for injection or implantation into a patient using an enzyme. For example, hematopoietic stem cells can be isolated from the matrix using collagenase or a solution thereof. Additional enzymes useful for isolation of cells from the matrix include, for example, proteases such as serine proteases, thiol proteases, and metalloproteinases, including the matrix metalloproteinases such as the collagenases, gelatinases, stromelysins, and membrane type metalloproteinase, or combinations thereof.

In any of the embodiments described herein, the collagen used herein may be any type of collagen, including collagen types I to XXVIII, alone or in any combination. In one embodiment, a mixture of type I and type III collagen is used. In one illustrative embodiment, the type III collagen can enhance differentiation into myeloid and lymphoid lineages and can enhance the proliferation of the hematopoietic stem cells seeded on the engineered collagen matrices.

In any of the embodiments described herein, hematopoietic stem cells can be suspended in a liquid-phase, collagen formulation designed to polymerize in situ to form a three-dimensional matrix. The formulation can comprises soluble collagen, for example, soluble type I collagen, and defined polymerization reaction conditions to yield engineered collagen matrices with controlled molecular composition, fibril microstructure, and mechanical properties (e.g., stiffness), for example. Matrix stiffness and fibril density can predictably modulate hematopoietic stem cell behavior.

Applicants have developed type I collagen formulations derived from various collagen sources, e.g., pig skin. These formulations comprise both type I collagen monomers (single triple helical molecules) and oligomers (at least two monomers covalently crosslinked together). The presence of oligomers enhances the self-assembly potential by increasing the assembly rate and by yielding three-dimensional matrices with distinct fibril microstructures and increased mechanical integrity (e.g., stiffness).

In any of the embodiments described herein, the engineered collagen matrix can have a predetermined percentage of collagen oligomers based on total isolated collagen added to make the engineered matrix. In various embodiments, the predetermined percentage of collagen oligomers can be about 0.5% to about 100%, about 30% to about 100%, about 40% to about 100%, about 50% to about 100%, about 60% to about 100%, about 70% to about 100%, about 80% to about 100%, about 90% to about 100%, about 95% to about 100%, or about 100%. In yet another embodiment, the collagen oligomers are obtained from a collagen-containing source material enriched with collagen oligomers (e.g., pig skin).

In any of the embodiments described herein, the engineered collagen matrices can have an oligomer content quantified by average polymer molecular weight (AMW). As described herein, modulation of AMW can affect polymerization kinetics, fibril microstructure, molecular properties, and fibril architecture of the matrices, for example, interfibril branching, pore size, and mechanical integrity (e.g., matrix stiffness). In another embodiment, the oligomer content of the purified collagen, as quantified by average polymer molecular weight, positively correlates with matrix stiffness.

In any of the embodiments described herein, monomer-rich collagen matrices can have an AMW of about 100 to about 280 kDa, about 250 to about 280 kDa, or about 250 to about 300 kDa, e.g., about 282 kDa. In another illustrative embodiment, oligomer-rich collagen matrices have an AMW of greater than about 300 kDa, for example, the AMW of an oligomer-rich collagen matrix can be about 300 kDa to about 2.8 MDa, about 400 kDa to about 2.8 MDa, about 400 kDa to about 750 kDa, about 400 kDa to about 850 kDa, about 350 kDa to about 1.5 MDa, or about 350 kDa to about 2.0 MDa. In one embodiment, the oligomer-rich collagen matrices have an AMW of greater than about 2.8 MDa.

The following examples illustrate specific embodiments in further detail. These examples are provided for illustrative purposes only and should not be construed as limiting the invention or the inventive concept in any way.

### EXAMPLES

### Example 1

### Development of a Three-Dimensional Culture Model

### Collagen Isolation—

Type I collagen, comprising oligomers and monomers, was acid solubilized and purified from porcine skin according to a modified protocol from (Gallop, P. M. and S. Seifter, Preparation and properties of soluble collagens, Methods in Enzymology, 1963, p. 635-641, incorporated herein by reference). All type I collagen formulations were prepared from the dermis of market weight pigs. To prepare collagen, skin was harvested from pig immediately following euthanasia and was washed thoroughly with cold water. The skin was stretched out and pinned to a board and stored at 4° C. The hair was removed with clippers. The dermal layer of the tissue was isolated by separating and removing the upper epidermal layer and the lower loose fatty connective layers. This removal was readily achieved by scraping the tissue with a knife or straight razor. The tissue was maintained at 4° C.

The resulting dermal layer tissue was washed in water and then cut into small pieces (approximately 1 cm²) and was frozen and stored at −80° C. The frozen skin pieces were pulverized under liquid nitrogen using an industrial blender or cryogenic grinder. Oligomer collagen was prepared as described previously (Kreger et al., 2010, incorporated herein by reference).

Soluble proteins were removed by extracting the pig skin powder (0.125 g/ml) with 0.5M sodium acetate overnight at 4° C. The resulting mixture was then centrifuged at 2000 rpm (700×g) at 4° C. for 1 hour. The supernatant was discarded and the extraction procedure repeated three additional times. The resulting pellet was then suspended (0.25 g/ml) in cold MilliQ water and then centrifuged at 2000 rpm (700×g) at 4° C. for 1 hour. The pellet was then washed with water two additional times. Collagen extraction was then performed by suspending the pellet (0.125 g/ml) in 0.075M sodium citrate. The extraction was allowed to proceed for 15-18 hours at 4° C. The resulting mixture was centrifuged at 2000 rpm (700×g) at 4° C. for 1 hour. The supernatant was retained and stored at 4° C. The pellet was re-extracted with 0.075M sodium citrate. The extraction process was repeated such that the tissue was extracted a total of three times. The resulting supernatants were then combined and centrifuged at 9750 rpm (17,000×g) at 4° C. for 1 hour to clarify the solution. The supernatant was retained and the pellet discarded.

Collagen was then precipitated from the supernatant by dialyzing (MWCO 12-14,000) extensively against 0.02 M disodium hydrogen phosphate at 4° C. The resulting suspension was then centrifuged at 2000 rpm at 4° C. for 1 hour and the pellet retained. The pellet was then resuspended and rinsed in cold MilliQ water. The suspension was centrifuged at 2000 rpm at 4° C. for 1 hour. The water rinse procedure was repeated two additional times. The resulting collagen pellet was dissolved in 0.1M acetic acid and then lyophilized. The lyophilized material was stored within a dessicator at 4° C. for use in engineering collagen matrices.

Selective polymerization in the presence of glycerol was used to further fractionate the pig skin collagen into oligomer-rich formulations as described previously (Na G. C., Biochemistry, 1989; 28(18):7161-7, incorporated herein by reference). A single source was obtained by performing the glycerol separation on isolated collagen obtained from a single pig hide. A pooled source was obtained from two collagen isolation batches from each of three separate pigs. Viscoelastic properties of polymerized matrices were measured in both oscillatory shear and unconfined compression on a stress-controlled AR2000 rheometer (TA Instruments, New Castle, Del.) using a stainless steel 40 mm diameter parallel plate geometry as described previously (Kreger S. T. et al., Matrix Biol, 2009; 28(6):336-46, incorporated herein by reference). Compared to the monomer-rich fraction, the oligomer-rich fraction showed a sharp increase in G′ and Ec as a function of concentration while maintaining a consistently low δ. An increase in G′, decrease in δ, and surprising increase in Ec was observed as illustrated in the top three panels of FIG. 12. These results indicate that changes in the fibril microstructure-mechanical properties observed with increased AMW are primarily due to increased interfibril cross-linking.

FIGS. 11 and 12 show that the inclusion of oligomers significantly enhances the polymerization rate and yields fibril microstructure-mechanical relationships with enhanced mechanical integrity. Matrices showed an increase in average projected poresize and no substantial change in fibril diameter and density as oligomer content (AMW) increased.

Reduced collagens were processed to eliminate reactive aldehydes generated from acid-labile cross-links. Neutral-buffered solutions of collagen oligomer (1 mg/ml) were chemically reduced by stirring with sodium borohydride (1 mg/10 mg collagen). Fresh sodium borohydride was added at 30-minute intervals for a total reduction time of 90 minutes (Gelman, R. A., Williams, B. R. and Piez, K. A. Collagen Fibril Formation: Evidence for a Multistep Process. J. Biol. Chem. 1979, 254:180-186, incorporated herein by reference). Reduced collagen solutions were then dialyzed extensively against 0.1M acetic acid and lyophilized. All collagens were dialyzed extensively against 0.1M acetic acid and then lyophilized. Prior to use, lyophilized collagens were dissolved in 0.01 N HCl. For cell studies, collagens were rendered aseptic by exposure to chloroform overnight at 4° C. Collagen concentration was determined using a Sirius Red (Direct Red 80) assay as known in the art.

Sodium dodecyl sulfate polyacrylamide gel electrophoresis (SDS-PAGE) was used to assess the purity and molecular composition of each collagen source. 12% Novex Tris-Glycine gels (Invitrogen, Carlsbad, Calif.) were used for identification of non-collagenous proteins and small molecular weight contaminants. SDS-PAGE in interrupted and uninterrupted formats and western blot analysis using mouse monoclonal antibodies specific for type I (AB6308, Abcam, Cambridge, Mass.) and type III (MAB 1343, Chemicon, Temecula) collagen were used for analysis of collagen type content (e.g. types I, III, and V). Gels were stained with Coomassie Blue (Sigma-Aldrich) or silver nitrate and imaged using a digital camera and light box. An alcian blue assay was used to assess sulfated glycosaminoglycan (GAG) content. Heparin derived from porcine intestinal mucosa (Sigma-Aldrich) was used to prepare a standard curve (1-20 heparin units/ml).

### Preparation of Three-Dimensional Collagen Matrices—

Collagen matrices were polymerized at a collagen concentration of 0.7 mg/ml. All collagen preparations were polymerized under identical reaction conditions to produce three-dimensional matrices as described previously (Kreger et al., 2010, incorporated herein by reference). Collagen solutions were diluted with 0.01 N HCl and neutralized with 10× phosphate buffered saline (PBS, 1×PBS had 0.17 M total ionic strength and pH 7.4) and 0.1 N sodium hydroxide to achieve neutral pH (7.4). Neutralized collagen solutions were kept on ice prior to the induction of polymerization by warming to 37° C. Due to the increased viscosity of collagen solutions, positive displacement pipettes (Microman, Gilson, Inc., Middleton, Wis.) were used to accurately pipette all collagen solutions.

Briefly, collagen was polymerized by using a 10×PBS, pH 7.4 solution consisting of 1.37M NaCl, 0.027M KCl, 0.081M Na₂HPO₄, 0.015M KH₂PO₄, 5 mM MgCl₂, and 1% w/v glucose. To polymerize collagen, a mixture was made of 1 ml of collagen in 0.01N HCl, 150 μl 10×PBS, pH 7.4, 150 μl 0.1N NaOH, 100 μl 13.57 mM CaCl₂, and 100 μl 0.01 N HCl. The composition was mixed well after each component was added. Polymerization was allowed to proceed at 37° C.

### Example 2

### Interaction of Hematopoietic Stem Cells and Osteoblasts

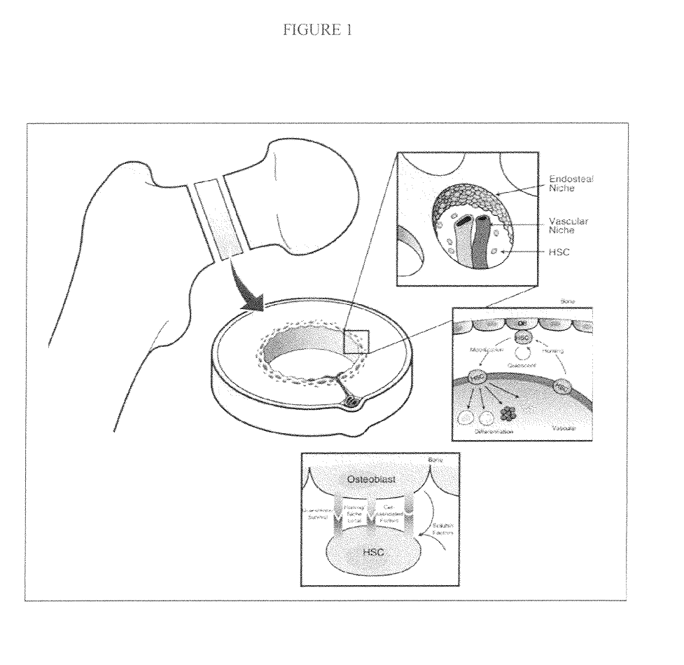

Hematopoietic stem cells (HSC) are located within the cancellous region of bones, and interact closely with osteoblasts [3] (FIG. 1). Murine bone marrow was harvested from long leg bones. Low-density marrow was isolated using a Ficoll separation, and the low-density cells were further isolated by flow cytometry. Cells exhibiting a Lin⁻Sca1⁺c-Kit⁺ (LSK) phenotype were identified to be early HSC progenitors, and were cultured in either the presence or absence of murine osteoblasts (OB) within three-dimensional (3D) engineered collagen type I matrix constructs. Matrix parameters, including collagen fibril density (9-20%) and shear storage modulus (50-800 Pa, G′) were systematically varied and quantified. Proliferation, clonogenic potential, and cell surface marker expression of LSK cells were measured after 7 days in culture.

### Example 3

### Preparation of Osteoblasts and LSK-OB Co-Culture

### Preparation of Osteoblasts—

Osteoblasts (“OB”) were freshly isolated from 2-day (2d) old C57BL/6 mice by sequential enzymatic digestion (Ciovacco W. A. et al., Bone, 2009; 44(1):80-86; Kacena M. A. et al., Journal of Histotechnology, 2004; 27:119-130, incorporated herein by reference). Calvariae from C57BL/6 mice less than 48 hours old were dissected, pretreated with EDTA in PBS for 30 min then subjected to sequential collagenase digestions (200 U/mL). Fractions 3-5 (collected between 45-60, 60-75, and 75-90 min through the digestion) were collected and used as OB. These cells are >95% OB or OB precursors as previously demonstrated.

### OB-LSK Co-Culture—

LSK (625 cells) from BoyJ mice (CD45.1) were seeded alone or in the presence of freshly isolated calvarial OB (25,000 cells) from C57Bl/6 mice (CD45.2) within collagen matrices prepared with G′ values of 50 Pa, 200 Pa, and 800 Pa (0.5 ml/well of 24-well plate). Parallel experiments were set in two-dimensions on tissue culture plastic and involved seeding densities of 500 LSK/well and 20,000 OB/well within a 24-well plate. Cultures were maintained for one week in medium consisting of 1:1 mix of IMDM and αMEM supplemented with 10% FBS, 1% Pen/Strep, and 1% L-Glutamine. All cultures were supplemented with a cocktail of cytokines containing recombinant murine SCF & IL3 (10 ng/mL), IGF1 & TPO (20 ng/mL), IL6 & Flt3 (25 ng/mL) and OPN (50 ng/mL) on day 0 and every 2 days thereafter. Cells were harvested on day 7 using enzymatic methods and counted. Fold increase in the number of cells derived from LSK cells was calculated relative to day 0 count.

### Example 4

### Hematopoietic Stem Cell (HSC)

### OB Medium

### Cell Types:

LSK=Lineage⁻Ska1⁺cKit⁺ mouse bone marrow-derived hematopoietic stem cells

OB=Mouse osteoblast cells

### Media:

In all experiments, the media used was:
1:1 mix of Iscove's Modified Dulbecco's Media (IMDM) and Modified Eagle's medium (MEM)
Supplemented with 10% Fetal Calf Serum (FCS), 1% Penicillin/Streptomycin, and 1% L-Glutamine
Cytokines:
   Stem cell factor (SCF)—10 ng/mL
   Interleukin 3 (IL3)—10 ng/mL
   Insulin-like growth factor 1 (IGF1)—20 ng/mL
   Thrombopoietin (TPO)—20 ng/mL
   Interleukin 6 (IL6)—25 ng/mL
   FMS-related tyrosine kinase 3 ligand (FLT3)—25 ng/mL
   Osteopontin (OPN)—50 ng/mL
      Experiments were performed in 24-well plates.

### Example 5

### Cell Harvest from Tissue Constructs

Cells were isolated from three-dimensional tissue constructs using enzymatic or non-enzymatic dissolution of the matrix. Enzymatic digestion involved incubation of tissue constructs in complete medium containing 500 U/ml collagenase (Worthington, Type IV) and 2.4 U/ml dispase for 20 minutes at 37° C. Following digestion, an equal volume of complete medium was added and the cell suspension centrifuged at 1000 rpm for 5 minutes. The pellet was washed in complete medium and then treated with 100 ul TrypLE (Gibco) for 15 minutes at 37° C. The cell suspension was diluted in complete medium, centrifuged to concentrate, and resuspended in complete medium.

If tissue constructs exhibit temperature-dependent matrix to solution properties, cells can alternatively be isolated in ice-cold cell harvest buffer containing 1 mM EDTA, 10% w/v glucose in phosphate buffered saline, pH 7.4. Constructs in cell harvest buffer can be maintained at 4° C. for 10 minutes with periodic agitation and then centrifuged at 1000 rpm for 5 minutes. The cell pellet can be redissolved in complete medium.

### Example 6

### Progenitor Cell Assay

Cells were plated in duplicate in 3 cm Petri dishes containing 1 ml methyl-cellulose with cytokines (MethoCult GF M3434, Stem Cell Technologies, Vancouver, BC). Cultures were maintained at 37° C. in humidified incubator at 5% CO₂ and colonies were counted on an inverted microscope after 7-days.

### Example 7

### Impact of Collagen Matrix Physical Properties on Proliferation

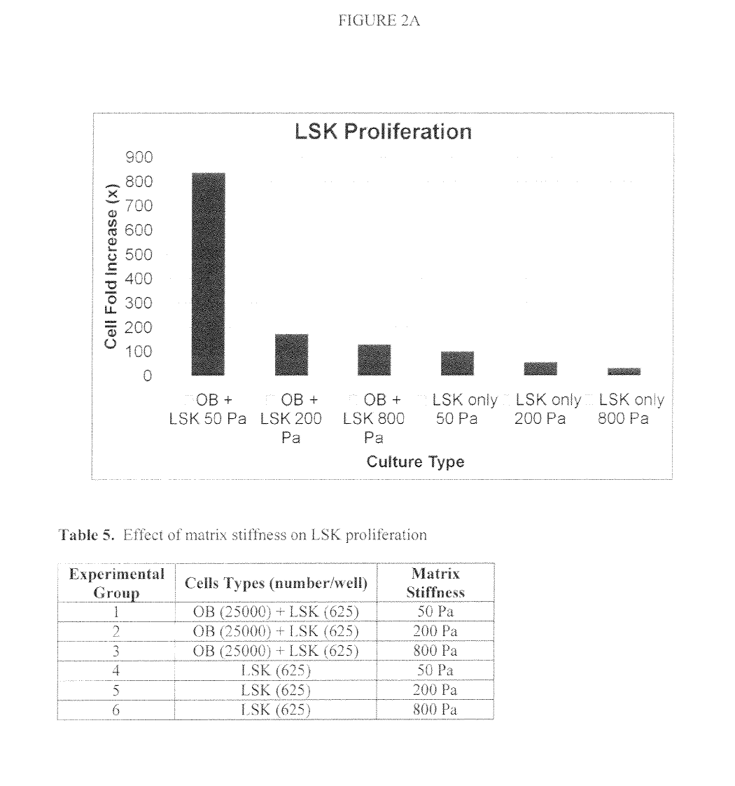

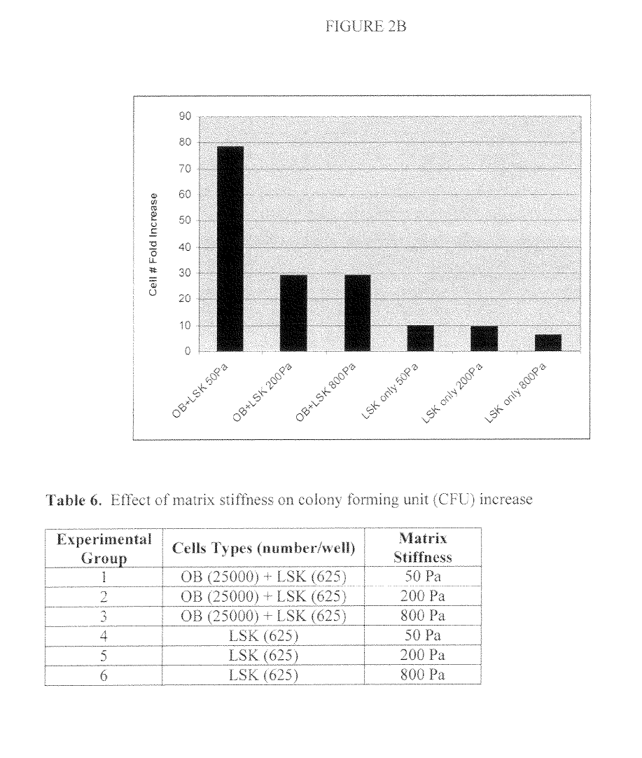

LSK cells were grown on three-dimensional engineered collagen type I matrix constructs having a shear storage modulus of 50 Pa, 200 Pa, and 800 Pa, in the presence and absence of OB. Cell proliferation was determined by performing a direct cell count on cells harvested from culture plates or matrices. A collagenase-based cocktail was used to harvest the cells from the matrices. LSK proliferation was shown to be inversely related to matrix stiffness. This trend is seen in both the presence and absence of OBs. FIG. 2A shows LSK proliferation (Table 5). FIG. 2B shows colony forming unit fold increase (Table 6). The greatest cell number increase occurs with matrices of low stiffness (e.g. 50 Pa). Furthermore, OBs increase proliferation at all levels of matrix stiffness (n=2).

### Example 8

### Impact of Collagen Matrix Physical Properties on Clonogenic Potential

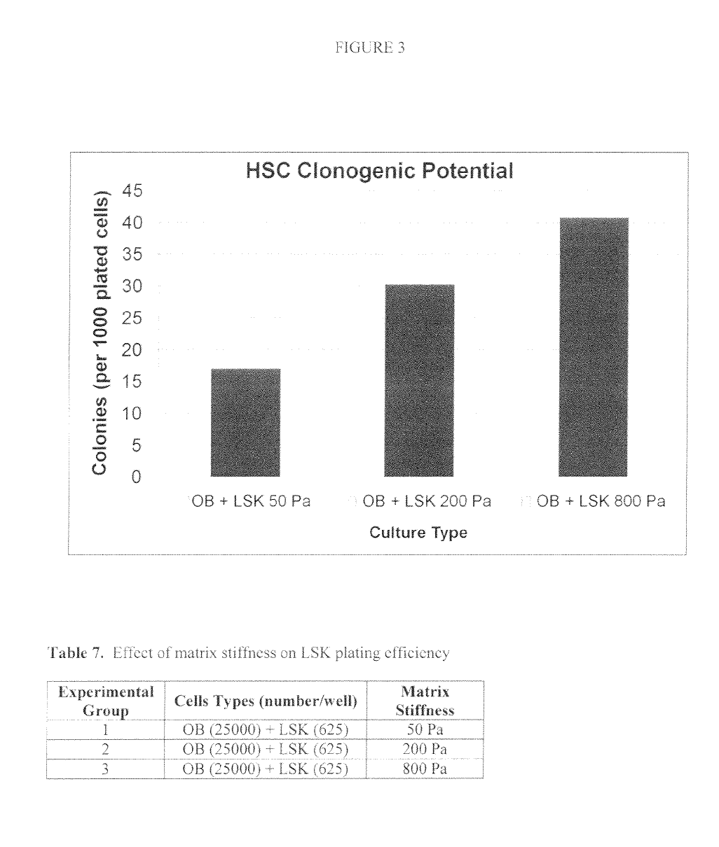

LSK cells were grown on three-dimensional engineered collagen type I matrix constructs having a shear storage modulus of 50 Pa, 200 Pa, and 800 Pa, in the presence of OB. Clonogenic potential was measured using a methyl cellulose assay as previously described (Orschell-Traycoff C M, Hiatt K, Dagher R N, Rice S, Yoder M C, Srour E F. Homing and engraftment potential of Sca1+lin− cells fractionated on the basis of adhesion molecule expression and position in cell cycle. Blood. 2000; 96:1380-1387, incorporated herein by reference). Cells were cultured for 7 days (n=2). The clonogenic potential of LSK cells was shown to be directly related to matrix stiffness (FIG. 3; Table 7).

### Example 9

### Impact of Collagen Matrix Physical Properties on Phenotype Maintenance

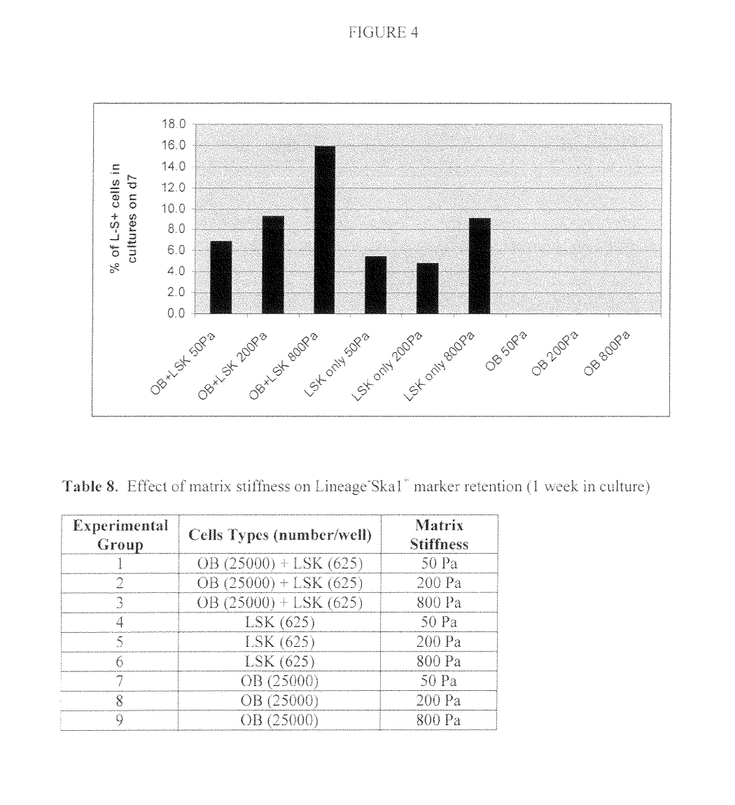

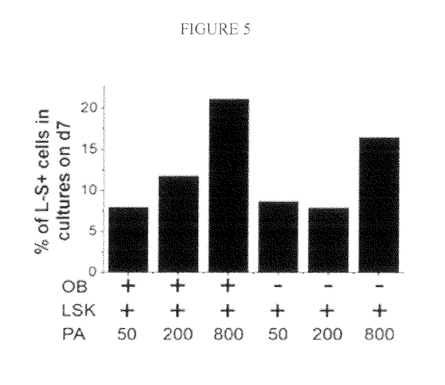

LSK cells were grown on three-dimensional engineered collagen type I matrix constructs having a shear storage modulus of 50 Pa, 200 Pa, and 800 Pa, in the presence and absence of OB. Lin⁻Sca1⁺ phenotype maintenance was determined by analyzing the percentage of Lin⁻Sca1⁺ cells harvested from the matrices. Lin⁻Sca1⁺ phenotype maintenance was enhanced in higher stiffness matrices. The presence of OB also promoted phenotype maintenance (n=2) (FIGS. 4 and 5; Table 8).

### Example 10

### Evaluation of Extracellular Matrix Components on Collagen Matrix Physical Properties

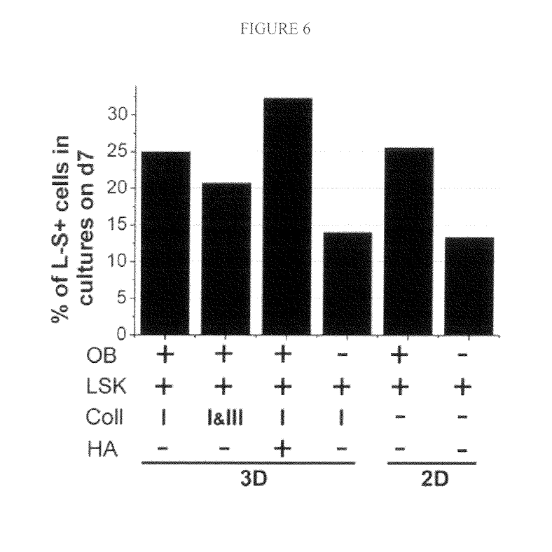

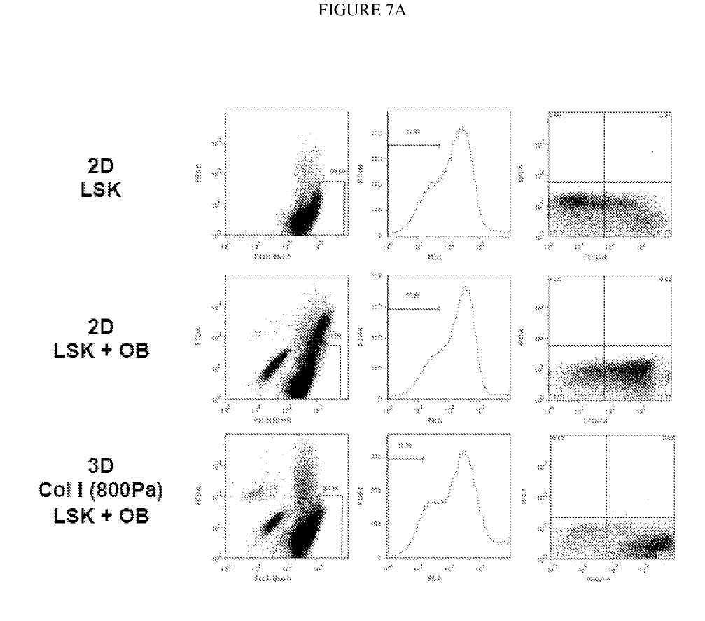

Studies were also conducted with more complex three-dimensional collagen-based matrices in which specific extracellular matrix (ECM) components were co-polymerized with type I collagen. For these studies, hyaluronic acid (HA) and type III collagen were selected based upon their prominence within the bone marrow ECM. Co-polymerization of type I collagen with increasing HA does not affect the fibril microstructure of the matrix but alters matrix viscoelasticity. However, co-polymerization of collagen types I and III result in matrices with decreased fibril diameter and increased fibril density. Compared to type I collagen matrices produced with the same total collagen content, type I+III matrices show a significant reduction in stiffness (E, G′). FIG. 6 shows the highest percentage of L-S+ cells retained in these cultures was observed in matrices containing HA. Thus, in addition to type I collagen, other bone marrow ECM components are critical for modulation of differentiation of primitive progenitor cells in three-dimensional cultures.

### Example 11

### Effect of Fibril Density and Matrix Stiffness Evaluated by Flow Cytometry

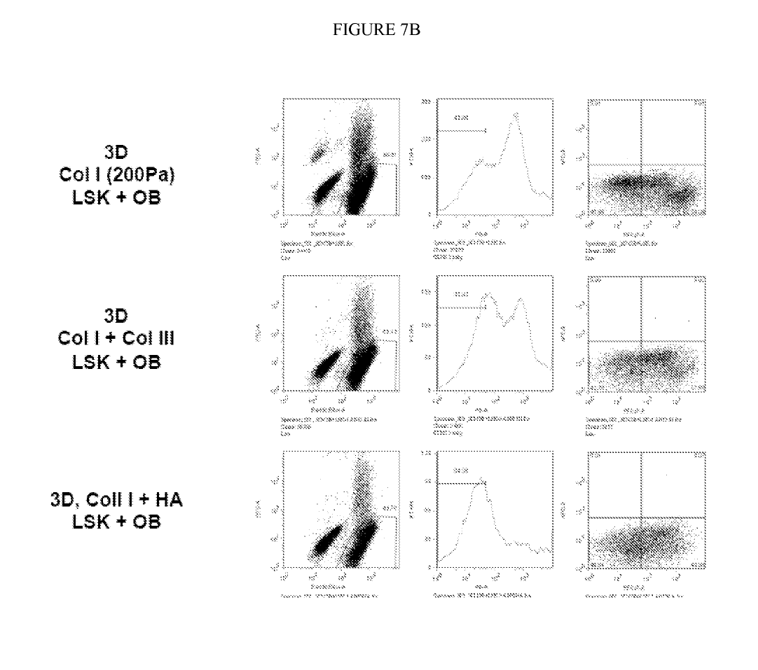

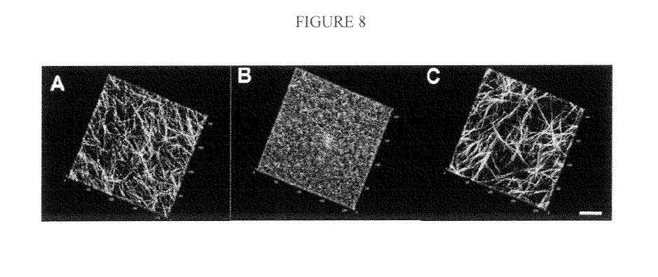

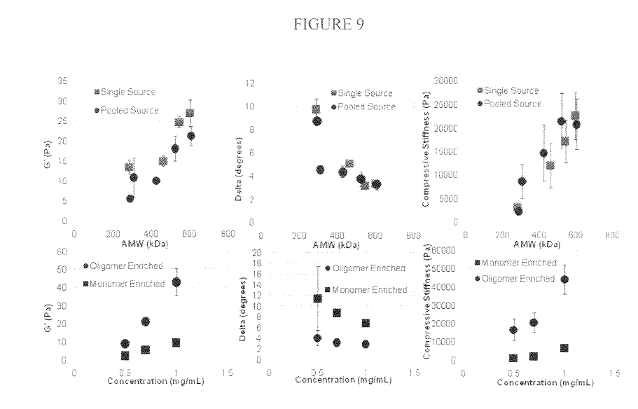

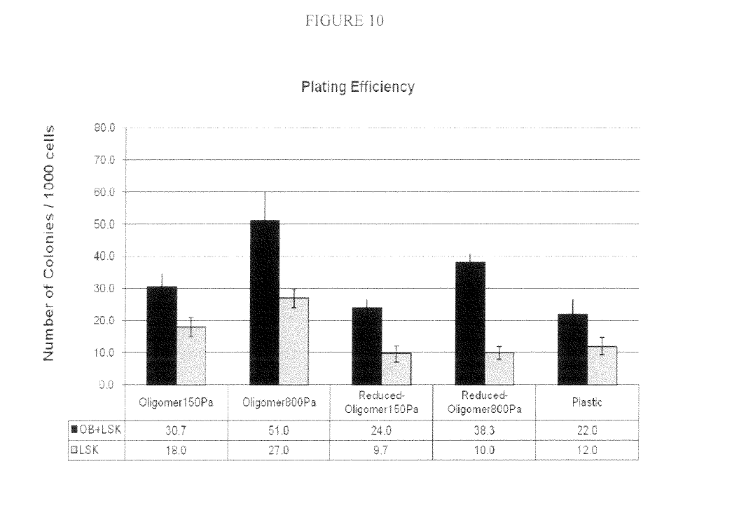

Flow cytometry results are shown in FIG. 7, illustrating the effect of fibril density and matrix stiffness on LSK proliferation, CFU fold increase, plating efficiency, and Lin-Sca1+ marker maintenance for: a 200 Pa LSK culture (7B, top panels); a traditional two-dimensional LSK culture (7A, top panels); a traditional two-dimensional culture with LSK+OB (7A, middle panels); an 800 Pa three-dimensional culture with LSK+OB (7A, bottom panels); a three-dimensional collagen type I and type III matrix with LSK and OB culture (7B, middle panels); and a three-dimensional collagen type I and hyaluranic acid matrix with an LSK and OB culture (7B, bottom panels). The second peak present represents a population of early hematopoietic progenitor cells. For 200 Pa matrices with hyaluronic acid (HA), HA was added at 1 mg/ml. For cell phenotyping by flow cytometry, cells were harvested from culture plates or matrices and immunolabeled with antibodies for specific cell surface markers.

### Example 12

### Plating Efficiency or Colony Forming Potential Provide a Functional Measure of HSC Progenitor Cell Function

The colony forming potential of LSK is dependent upon the in vitro culture format. LSK were cultured in the presence or absence of OB within three-dimensional collagen matrices or on plastic for 7 days. LSK progenitor cell function then was assessed using a well-established methyl cellulose assay. FIG. 13 shows that LSK cultured alone within Oligomer800 Pa showed significantly higher (p<0.05) colony forming potential compared to those on plastic. LSK cultured within all other collagen matrix formulations displayed colony forming potential statistically similar to those on plastic. Co-culture of LSK and OB significantly increased LSK colony forming potential for all culture formats. Colony forming potential was the greatest for LSK+OB cultured within oligomer and reduced-oligomer matrices prepared with a stiffness of 800 Pa. LSK cultured in the presence of OB within 150 Pa collagen matrices showed colony forming potential that was statistically similar to that observed on plastic. Therefore, the format of the in vitro culture influences the colony forming potential of LSK.

### Example 13

### Evaluation of In Vivo Bone Marrow Repopulating Potential

Three-dimensional tissue constructs were prepared by seeding LSK (625 cells/well) from C57Bl/6 mice (CD45.2) along with OB (25,000 cells/well; also from C57Bl/6 mice) on 50, 200, and 800 Pa collagen matrices. On day 10, all well contents were harvested using a collagenase/dispase cocktail and injected via the tail vein into a lethally irradiated (1,100 Rad, split dose) C57BL/6×BoyJ F1 recipient (CD45.2/CD45.1) plus 100,000 competitor low density BM cells from BoyJ mice (CD45.1). Five mice were transplanted per group. After 2 months, chimerism was assessed as CD45.2/(CD45.2+CD45.1)×100.

Results showed that the in vivo bone marrow repopulating potential was greatest for cells harvested from the 800 Pa construct. The level of chimerism or engraftment was 2-fold and 8-fold less for cells harvested from the 200 Pa and 50 Pa matrices, respectively.

### REFERENCES

All cited references are expressly incorporated herein by reference.
[1] Spradling A, Drummond-Barbosa D, Kai T. Stem Cells Find Their Niche. Nature, Volume 414, 2001.
[2] Scadden D T. The stem cell niche as an entity of action. Nature, Volume 441, 2006.
[3] Shiozawa Y, Havens A M, Pienta K J, Taichman R S. The bone marrow niche: habitat to hematopoietic and mesenchymal stem cells, and unwitting host to molecular parasites. Leukemia, Volume 22, Issue 5, 2008.
[4] Compston J E. Bone marrow and bone: a functional unit. Journal of Endocrinology, Volume 173, 2002.

TABLE 1
Shows 200 Pa Cultures (LSK Fold Increase)

| | Exp 2 | Exp 3 | Avr |
|---|---|---|---|
| 3D - 200PA | | | |
| OB | 0 | 0 | 0 |
| OB + LSK | 80 | 191 | 135.5 |
| LSK | 46 | 92 | 69 |
| OB + LSK (Type I and III) | 64 | 215 | 139.5 |
| OB + LSK (Type I and HA) | 66 | 268 | 167 |
| 2D | | | |
| OB | 0 | 0 | 0 |
| OB + LSK | 1181.25 | 1575 | 1378.13 |
| LSK | 750 | 1200 | 975 |

TABLE 2
Shows 200 Pa Cultures (CFU Fold Increase)

| | Exp 2 | Exp 3 | Avr |
|---|---|---|---|
| 3D - 200PA | | | |
| OB | 0 | 0 | 0 |
| OB + LSK | 36.7 | 33.8 | 35.25 |
| LSK | 11.2 | 9.56 | 10.38 |
| OB + LSK (Type I and III) | 37.7 | 28.9888 | 33.3444 |
| OB + LSK (Type I and HA) | 24.2 | 51.191 | 37.6955 |
| 2D | | | |
| OB | 0 | 0 | 0 |
| OB + LSK | 355.7 | 141.573 | 248.637 |
| LSK | 142 | 67.4157 | 104.708 |

TABLE 3
Shows 200 Pa Cultures (L-S+ of CD45.1)

| | Exp 2 | Exp 3 |
|---|---|---|
| 3D - 200PA | | |
| OB | 0 | 0 |
| OB + LSK | 24.98 | 6.74 |
| LSK | 14.01 | 1.67 |
| OB + LSK (Type I and III) | 20.72 | 1.51 |
| OB + LSK (Type I and HA) | 32.33 | 3.85 |
| 2D | | |
| OB | 0 | 0 |
| OB + LSK | 25.58 | 8.38 |
| LSK | 13.36 | 7.47 |

TABLE 4
Effect of select matrix molecules [collagen type III (0.5 mg/mL), hyaluronic acid
(1 mg/mL)]. The measured outcomes include: LSK Proliferation, LSK colony forming unit
(CFU) increase, and maintenance of Lineage⁻Ska1⁺ surface markers following 1 week culture

| Experimental | Cells Types | Matrix | Additional Matrix |
|---|---|---|---|
| Group | (number/well) | Stiffness | Molecules: |
| 1 | OB (25000) | 200 Pa | — |
| 2 | OB (25000) + LSK (625) | 200 Pa | — |
| 3 | LSK (625) | 200 Pa | — |
| 4 | OB (25000) + LSK (625) | 200 Pa | Type III Collagen |
| | | | (0.5 mg/ml) |
| 5 | OB (25000) + LSK (625) | 200 Pa | Hyaluronic Acid |
| | | | (1 mg/ml) |
| 6 | OB (25000) | 800 Pa | — |
| 7 | OB (25000) + LSK (625) | 800 Pa | — |
| 8 | LSK (625) | 800 Pa | — |
| 9 | OB (25000) + LSK (625) | 800 Pa | Type III Collagen |
| | | | (0.5 mg/ml) |
| 10 | OB (25000) + LSK (625) | 800 Pa | Hyaluronic Acid |
| | | | (1 mg/ml) |

2D Replicates:

| | | | |
|---|---|---|---|
| 11 | OB (20000) | — | — |
| 12 | OB (20000) + LSK (500) | — | — |
| 13 | LSK (500) | — | — |

1. A method for maintaining or proliferating hematopoietic stem cells in vitro, said method comprising the steps of:
contacting an hematopoietic stem cell population with an engineered collagen matrix; and
maintaining or proliferating the hematopoietic stem cells in vitro.

2. The method of claim 1 wherein the collagen matrix has a fibril density of about 5% to about 30%, and wherein the collagen matrix has a storage modulus of about 10 Pa to about 3200 Pa.

3. The method of claim 1 further comprising the step of contacting said matrix with osteoblasts.

4. The method of claim 3 wherein the osteoblasts enhance proliferation or maintenance of the hematopoietic stem cells.

5. The method of claim 1 wherein the matrix has a storage modulus of about 10 Pa to about 100 Pa.

6. The method of claim 1 wherein the matrix has a storage modulus of about 500 Pa to about 2000 Pa.

7. The method of claim 1 wherein the matrix has a storage modulus of about 700 Pa to about 1500 Pa.

8. The method of claim 1 wherein the matrix has a storage modulus of about 700 Pa to about 900. Pa.

9. The method of claim 8 wherein the hematopoietic stem cells are undifferentiated.

10.-11. (canceled)

12. The method of claim 8 wherein the hematopoietic stem cell population exhibits clonogenic potential.

13.-18. (canceled)

19. A composition comprising hematopoietic stem cells and an engineered collagen matrix.

20. The composition of claim 19 further comprising osteoblasts.

21. The composition of claim 19 wherein the collagen matrix has a fibril density of about 5% to about 30%, and wherein the collagen matrix has a storage modulus of about 10 Pa to about 3200 Pa.

22. The composition of claim 20 wherein the osteoblasts enhance proliferation or function of the hematopoietic stem cells.

23. The composition of claim 19 wherein the matrix has a storage modulus of about 10 Pa to about 100 Pa.

24. The composition of claim 19 wherein the matrix has a storage modulus of about 500 Pa to about 2000 Pa.

25. The composition of claim 19 wherein the matrix has a storage modulus of about 700 Pa to about 900 Pa.

26. The composition of claim 25 wherein the hematopoietic stem cells are undifferentiated.

27. The composition of claim 25 wherein the hematopoietic stem cell population exhibits expression of an hematopoietic stem cell marker.

28. (canceled)

29. The composition of claim 25 wherein the hematopoietic stem cell population exhibits clonogenic potential.

30.-64. (canceled)
